(19) United States
(12) Patent Application Publication
(10) Pub. No.: US 2010/0076501 A1
(43) Pub. Date: Mar. 25, 2010

(54) METHOD AND DEVICE FOR BONE REGENERATION

(75) Inventors: Adam S. Hacking, Montreal (CA); Edward J. Harvey, Montreal (CA)

Correspondence Address: DINSMORE & SHOHL LLP, FIFTH THIRD CENTER, ONE SOUTH MAIN STREET, SUITE 1300, DAYTON, OH, 45402-2023, US

(73) Assignees: Adam S. Hacking, Montreal (CA); Edward J. Harvey, Montreal (CA)

(21) Appl. No.: 11/871,633

(22) Filed: Oct. 12, 2007

Related U.S. Application Data
(60) Provisional application No. 60/829,340, filed on Oct. 13, 2006.

(51) Int. Cl. A61B 17/56; A61F 2/28

(52) U.S. Cl. 606/86 R; 623/23.5

(57) ABSTRACT

The invention is directed to a method for adapting a bone graft implantable into a host bone for promoting host bone regeneration, the method comprising providing a bone graft comprising bone material and having a surface contactable with a host bone; and adapting at least a portion of the surface to produce a surface roughness similar to the roughness of a fracture surface of bone. Also included is a bone graft having at least a portion of a bone graft surface with a surface roughness similar to that of a fracture surface of bone, a device for adapting bone material having a plurality of impact tips to contact a surface of the bone material to be adapted to produce a bone material surface roughness similar to the roughness of a fracture surface of bone, and use of a device to adapt a surface of a bone material, the device having an impact tip arranged to contact the surface of the bone material to adapt the surface to produce a surface roughness similar to the roughness of a fracture surface of bone.

### CROSS REFERENCE TO RELATED APPLICATIONS

The Applicants hereby claim priority from U.S. patent application No. 60/829,340 filed 13 Oct. 2006, the contents of which are hereby incorporated by reference in their entirety.

### FIELD OF THE INVENTION

The invention relates generally to a method for adapting bone and bone grafts, a bone graft, a device and a use of a device, all for promoting bone regeneration.

### BACKGROUND OF THE INVENTION

Damage to bone can occur through fracture, injury, disease or surgery and may affect any part of the skeletal system. Bone grafts are often used to assist in the repair or healing of damaged bone, for example in the fields of orthopaedics, maxillo-craniofacial, and periodontics. In cases of non-union or mal-union of bone fractures, for example, one or more bone grafts can be placed around the bone fracture site to promote bone healing. Bone grafting techniques are also used to repair skeletal defects resulting from tumour resection, bone loss associated with periodontal disease and osseointegration of prosthetic implants in the edentulous mandible, for example.

Bone graft (osteograft) materials may include natural or synthetic bone. Natural bone graft materials include cortical or cancellous bone material transplanted from a donor site to a host site. Autografts are grafts taken from the intended recipient of the graft. Allografts are grafts transplanted from a donor of the same species as the intended recipient but of different genetic makeup such as another live patient or a cadaver. Xenografts are grafts taken from a species different than the intended recipient.

Autografts harvested from the patient at the time of surgery are the standard for bone grafting. Autografts have been shown to promote bone growth at the site of grafting (osteoinductive), form new bone themselves (osteogenic) and provide a scaffold for bone ingrowth (osteoconductive). Advantageously, as autografts are harvested from the same patient in which they are to be implanted, there is little or no risk of ‘rejection’ of the graft or of transmitting viruses. However, a patient may have a limited supply of bone for graft and autografts can suffer from donor site morbidity.

Allografts and xenografts are available in greater supply than autografts. However, they are less osteoinductive, may induce a greater immunogenic response and suffer from a higher risk of disease transfer compared to autografts. Therefore, adjunct therapies are often used with allografts and xenografts to promote bone regeneration and repair. These include the use of biomaterials that promote cellular infiltration and osteogenesis and biologics such as bone morphogenetic proteins (BMPs), TGFb and PTH, which stimulate cell replication and activity. In addition, biologics which stimulate vascularization and remodeling of the allografts and xenografts may also increase graft effectiveness. While these adjuvant therapies increase graft remodeling and new bone formation, they are costly and include risk factors such as osteosarcoma, marrow fibrosis and ectopic bone formation.

The use of bone allografts and xenografts to augment bone healing is expensive and the failure rate is relatively high. Treatments involving bone grafts often require protracted hospital stays which further increase the associated costs and the burden on the medical system. Thus a need exists for a safer, more efficacious and cost effective method to promote bone regeneration, growth and repair with bone materials such as allografts and xenografts. There also exists a need to improve the working efficacy of autografts.

Therefore, it is desired to overcome or reduce at least some of the above-described problems.

### SUMMARY OF THE INVENTION

The present invention reduces the difficulties and disadvantages of the aforesaid designs and treatments. The Inventors made a surprising discovery that some of the physical characteristics (e.g. the roughness (Ra)) of the fracture surfaces of bone are similar regardless of the mode of fracture of the bone, and that such physical characteristics when applied to a bone material can stimulate bone regeneration at that site. The Inventors also surprisingly demonstrated that this bone regeneration was predominantly due to the physical surface characteristics and not primarily as a result of the chemical composition of bone. Thereby, bone material surfaces having some or all of these physical characteristics can be used to treat bone damage such as bone fractures or bone defects. The Inventors have discovered that this can be achieved by adapting a surface of bone material such as by selectively removing bone material from the surface. This is surprising, given the general teaching in the fields of orthopaedics and dentistry which is against the removal of the periosteum (tissue covering bone) or the adaptation of bone surfaces.

From one aspect, there is provided a bone graft having a surface contactable with a host bone for promoting regeneration of the host bone, wherein at least a portion of the surface has a surface roughness similar to that of a fracture surface of bone.

The bone graft surface includes macrostructures and microstructures, the macrostructures being peaks and having a peak-to-peak spacing which is substantially less than that of an unfractured bone surface. Preferably, the peak-to-peak spacing is less than about 180 μm. More preferably, the peak-to-peak spacing is between about 0.1 and about 180 μm, about 0.1 to 30 μm, about 0.5 to 30 μm or about 0.5 to 20 μm. Preferably, the peaks are randomly distributed across the bone graft surface.

The surface roughness of the bone graft, as defined by Ra, is more than about 0.1 μm. More preferably, the Ra is between about 0.1 to 400 μm, about 0.5 to 400 μm, about 0.1 to 20 μm or about 0.5 to 20 μm.

From another aspect, there is provided a method for adapting a bone graft implantable into a host bone for promoting host bone regeneration, the method comprising: providing a bone graft comprising bone material and having a surface contactable with a host bone; and adapting at least a portion of the surface to produce a surface roughness similar to the roughness of a fracture surface of bone.

The bone graft surface can be adapted to include macrostructures and microstructures, the macrostructures being peaks and having a peak-to-peak spacing which is substantially less than that of an unfractured bone surface. Preferably, the peak-to-peak spacing is less than about 180 μm. The bone graft surface can be adapted by selectively indenting bone material at the surface or selectively removing bone material from the surface, such as by mechanically contacting the surface with at least one impacting tip to adapt it.

The bone graft of the invention provides an improved rate of attachment to a host bone (faster fusion) by promoting or encouraging bone formation and regeneration to minimize the post-surgery healing period and the stay time in hospital. Also, the bone graft of the invention provide an increased chance of success and a mechanically strong bone with the host bone. Therefore, there is potential for less pain than that provided by bone grafts currently in orthopaedic use.

From yet another aspect, there is provided a method for adapting bone for promoting bone regeneration, the method comprising: accessing at least a portion of a surface of bone to be adapted, the bone comprising bone material; and adapting the surface to produce a surface roughness similar to the roughness of a fracture surface of bone. The bone surface can be adapted by selectively indenting bone material at the surface or selectively removing bone material from the surface. The bone surface can be adapted by mechanically contacting the surface with at least one impacting tip to adapt it. The bone surface is adapted to include macrostructures and microstructures, the macrostructures being peaks and having a peak-to-peak spacing which is substantially less than that of an unfractured bone surface. The peak-to-peak spacing is less than about 180 μm. The roughness, as defined by Ra, is less than about 400 μm. An application of this aspect of this invention is in regard to bone fractures. The bone surfaces near or adjacent the fracture site may be adapted according to the invention. This may be performed to enhance the rate and robustness of the bone healing response or as a prophylactic measure to avoid the chances of non-union or mal-union.

From a further aspect, there is provided a device for adapting bone material, the device comprising: a first head having a plurality of impact tips arranged thereon as an array to contact a surface of the bone material to be adapted to produce a bone material surface roughness similar to the roughness of a fracture surface of bone. The device can comprise an actuator to provide a reciprocating movement to the plurality of impact tips. The device can comprise an elongate arm having a first end to which the first head is attachable, the elongate arm being sized and shaped to access an inner surface of the bone. In another embodiment, the device includes a second head having a plurality of impact tips arranged thereon as an array to contact a surface of the bone material to be adapted to produce a bone material surface roughness similar to the roughness of a fracture surface of bone, the second head being attachable to the elongate arm. The first and second heads can be arranged to be moveable towards and away from each other whilst remaining attachable to the elongate arm. The impact tips may have a diameter of from about 0.5 μm to about 25.0 μm and a tip separation of from about 0.1 μm to about 25.0 μm.

From a yet further aspect, there is provided use of a device to adapt a surface of a bone material, the device having an impact tip arranged to contact the surface of the bone material to adapt the surface to produce a surface roughness similar to the roughness of a fracture surface of bone. The impact tip can have a diameter of from about 0.5 μm to about 25.0 μm, preferably about 20.0 μm. The device further comprises an actuator to provide a reciprocating movement to the impact tip.

By means of the invention, the treatment of damaged bone such as bone fracture and defects will be easier and cheaper. For example, the method of the invention provides an adapted bone material surface for promoting bone regeneration which is simple, effective and not damaging to the structural integrity of the bone material itself. Advantageously, the surface can be adapted (textured) without the use of complex apparatus or devices.

The embodiments of the invention are envisaged to have application in various orthopedic procedures, such as cranio-facial, maxillo-facial procedures and long bone fracture, mal-union, non-union surgical procedures, and bone defect repair including defects from surgical incisions and disease for preservation or repair of the bone, and implant fixation, for example. Also, to aid in the attachment of soft tissue, such as ligaments and tendons, to bone.

### BRIEF DESCRIPTION OF THE DRAWINGS

Further aspects and advantages of the present invention will become better understood with reference to the description in association with the following in which:

FIG. 1A defines the terms “peak-to-peak spacing”, “peak-to-valley height” and “peak diameter”, and FIG. 1B defines the term “Ra”;

FIG. 2 is a schematic illustration of a bone graft according to an embodiment of the present invention;

FIG. 3 is a schematic illustration of a device, having a head, according to an embodiment of the present invention;

FIG. 4A is a schematic illustration of a side view of the head of FIG. 3, and FIG. 4B is a schematic illustration of a bottom view of the head of FIG. 3;

FIG. 5 is a schematic illustration of a bottom view of another embodiment of the head of FIG. 3;

FIG. 6A is a schematic illustration of another embodiment of the device of FIG. 3;

FIG. 6B is a schematic illustration of yet another embodiment of the device of FIG. 3;

FIG. 7 shows SEM pictures (1000×), from Example 1, of bone fracture surfaces from A) transverse, B) oblique, C) comminuted, D) butterfly fracture modes;

FIG. 8 shows non-contact optical profiles of the bone fracture surfaces of FIG. 7 (magnification 102×);

FIG. 9 shows SEM pictures (1000×) of bone surfaces from A) and C) endosteal, and B) and D) periosteal cortices from Example 1;

FIG. 10 shows SEM pictures (1000×), from Example 2, of bone fracture surfaces from A) mouse, B) rabbit, C) rat and D) canine;

FIG. 11 shows SEM pictures (1000×), from Example 2, of endosteal bone surfaces from A) mouse, B) rabbit, C) rat and D) canine A);

FIG. 12 shows cell culture disks, from Example 3, having A) a textured surface, B) a fractured surface, and C) a polished surface;

FIG. 13 shows optical profilometry scans of the cell culture disks of FIG. 12 having A) a textured surface, B) a fractured surface, and C) a polished surface;

FIG. 14 SEM pictures (500×) of the surfaces of the cell culture disks of FIG. 12 having A) a textured surface, B) a fractured surface, and C) a polished surface;

FIG. 15 illustrates the bone regeneration (forming) effect of a cortical bone adapted according to an embodiment of a method of the present invention;

FIG. 16 illustrates the bone regeneration effect of a xenograft according to an embodiment of the present invention; and

FIG. 17 illustrates the bone regeneration effect of an allograft according to an embodiment of the present invention.

### DETAILED DESCRIPTION OF THE INVENTION

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including”, “comprising”, or “having”, “containing”, “involving” and variations thereof herein, is meant to encompass the items listed thereafter as well as, optionally, additional items. In the following description, the same numerical references refer to similar elements.

As used herein, the term “bone” includes the whole or any part of natural bone anywhere in a body of an animal, such as a human.

As used herein, the term “bone graft” includes the whole or any part of a graft derived from natural bone, such as cortical or cancellous bone. The term includes autografts, allografts or xenografts which may or may not be fully or partially demineralized.

As used herein, the term “bone material” includes any or all of the material making up bone, such as bone mineral matrix and intercellular bone tissue substance.

As used herein, the term “host bone” refers to a bone site in an intended recipient (host) of a bone graft. The bone site may be near or at a bone fracture, bone defect, bone cut or any other type of bone damage.

As used herein, the term “bone fracture surface” refers to the fracture surface of compact bone between the endosteal or periosteal surfaces.

As used herein, the terms “macroroughness”, “macrotexture” or “macrofeatures” refer to larger surface features within the micron range. These larger surface features include peaks which have smaller surface features (“microfeatures”, “microroughness” or “microtexture”) superimposed thereon.

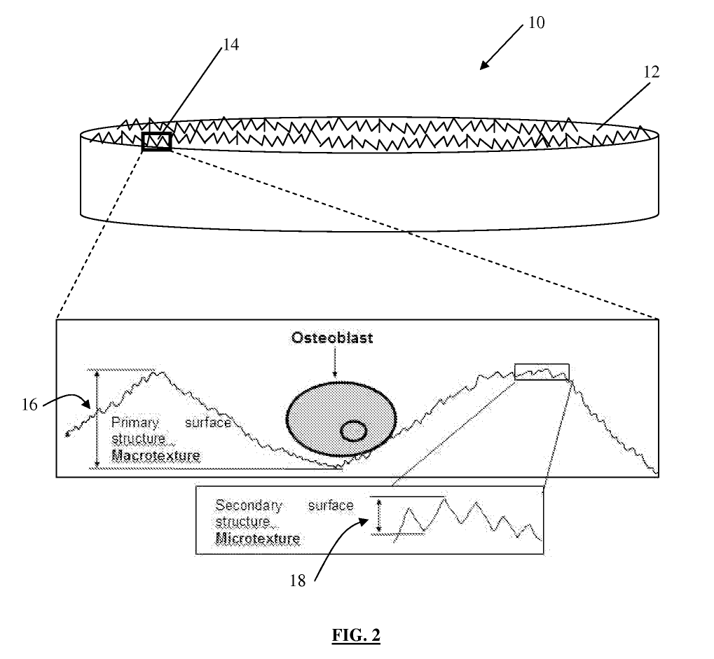

As used herein, the terms “microroughness”, “microtexture” or “microfeatures” refer to smaller surface features within the micron scale and which are smaller than the macrofeatures. The terms “macrotexture” and “microtexture” are illustrated in FIG. 2.

The term “texture” or “roughness”, as used herein, is meant to encompass both the microtexture and the macrotexture of a surface within the micron range.

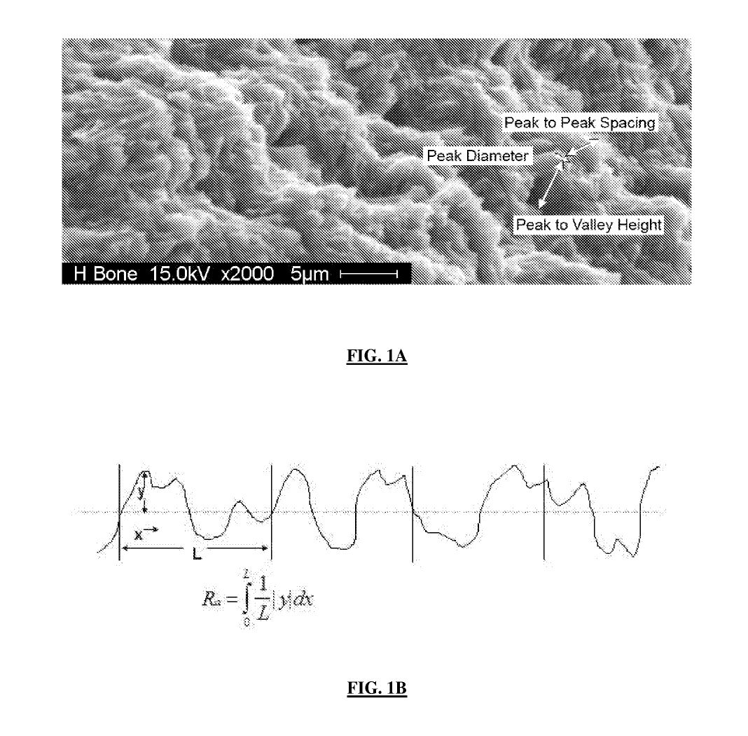

The terms “mean roughness (Ra)”, “root-mean-square roughness (Rq)”, peak-to-peak spacing, peak-to-valley height and peak diameter are parameters for defining the texture or roughness of a surface and are defined in FIG. 1. Mean roughness (Ra) is defined as the average deviation from the mean centerline roughness of the surface (macrotexture and microtexture). It is quantified using atomic force microscopy or white light interferometry in ways known to a person skilled in the art.

The root-mean-square roughness (Rq) is defined as the root-mean-square deviation of the profile from the mean line over one sampling length. The peak-to-peak spacing is defined as the shortest distance between adjacent peaks as manually measured from SEM photographs of the surface. The peak-to-valley height is defined as the distance from a base of the peak to a tip of peak as manually measured from SEM photographs of the surface. The peak diameter is defined as the longest distance measurable along the tip of a peak as manually measured from SEM photographs of the surface.

The present invention relates to a finding by the Inventors that a bone material surface which has been adapted so that it has roughness characteristics resembling at least some of those of a fracture surface of bone, will promote bone regeneration, either at the adapted surface or at another bone surface in contact with the adapted bone surface. By bone regeneration it is meant any kind or stage of bone growth such as repair and healing.

Referring to FIG. 2, there is provided a bone graft 10 having a surface 12 contactable with a host bone (not shown) for promoting regeneration of the host bone or osseointegration with the host bone, wherein at least a portion of the surface 12 has a surface roughness 14 similar to that of a fracture surface of bone. The roughness or texture of fracture surfaces of bone have been characterized by the Inventors, and compared with the texture of unfractured (smooth) surfaces of bone (see Examples 1 and 2).

The bone graft surface roughness 14, within the micron range, comprises macrofeatures (a primary structure) 16, also referred to as peaks, and microfeatures (a secondary structure) 18. The microfeatures are smaller than the macrofeatures and are applied to, or superimposed on, the surface of the macrofeatures 16. The peaks are arranged or distributed across the surface 12 in a random or un-orientated manner. In other words, the arrangement of the peaks does not form a regular pattern such as striations. The average peak-to-peak spacing is substantially less than that of smooth surfaces of bone. Preferably, the average peak-to-peak spacing of the surface 12 is less than about 180 μm. More preferably, the average peak-to-peak spacing of the surface 12 is less than about 30 μm, between about 0.1 and 30 μm, between about 0.5 and 30 μm, or between about 0.5 and 20 μm. The average peak-to-valley height is substantially more than that of smooth surfaces of bone. The average peak-to-valley height is preferably more than about 1 μm. More preferably, the average peak-to-valley height is between about 1 and 15 μm, about 1 and 10 μm, or about 1 and 5 μm. The average peak diameter is less than about 140 μm. More preferably, the average peak diameter is between about 0.1 and 20 μm, about 0.1 and 15 μm, or about 0.1 and 10 μm.

The surface 12 has a roughness, as defined by Ra, which is substantially more than that of smooth (unfractured) bone. Preferably, the Ra is more than about 0.1 μm. More preferably, the Ra is between about 0.1 to 400 μm, about 0.5 to 400 μm, about 0.1 to 20 μm or about 0.5 to 20 μm.

The bone graft may comprise any part or parts of cortical and/or cancellous bone, and may be an allograft, an autograft or a xenograft. The bone graft may also comprise a portion of an allograft, an autograft or a xenograft.

In use, the bone graft 10 is implanted at a site in a host animal or human patient where bone healing, supplementation, regeneration (formation), or enhanced bone formation is required. This may be at a damaged bone site such as at, or by, a bone fracture or crack, a bone defect, an excised bone or other bone material site. When the surface 12 is implanted adjacent a bone site, osseointegration of the bone graft with the host bone is enhanced or promoted by virtue of the surface texture of the bone graft 10. This may also be at a site of implantation to enhance boney fixation to the implant.

The bone graft 10 of the invention is prepared by adapting at least a portion of a surface of any available or conventional bone graft to produce a surface roughness similar to the roughness of a fracture surface of bone, as defined above. A conventional bone graft is obtained in a manner known in the art, or in any other way, before a portion of its surface is adapted. For example, an allograft can be obtained from a bone bank and adapted according to the method of the invention before being sterilized, such as with steam or gamma irradiation, and implanted.

The method of adapting the bone graft surface includes selectively indenting bone material and/or selectively removing bone material from the surface whilst maintaining the structural integrity of the bone graft to achieve the desired surface roughness. This is preferably achieved by mechanically working or contacting selected portions across the surface with at least one impacting tip, or an array of impacting tips. The impacting tip or tips should have a diameter suitable for producing the desired roughness, preferably ranging from about 0.5 to about 25 μm.

In a preferred embodiment, the surface is selectively worked by contacting the impact tip on different locations on the surface. Then the orientation of the surface is changed relative to the impact tip or tips by rotating the surface in the same plane, such as by 30°, and the selective mechanical working repeated. This helps to provide the random or irregular surface roughness or macrotexture on the bone graft surface. It will be appreciated that by selectively impacting or removing material as is required, the removal of an excess of bone material is avoided and the structural integrity of the bone graft is maintained.

In an alternative embodiment, the method of adapting the bone graft surface is performed by acid etching, electroetching, electropitting, mechanical abrasion (e.g. burr, wire wheel), particulate abrasion by any hard material (sand blasting), deformation of the surface by freezing, deformation of the surface by high pressure water with or without an abrasive, any type of lithography and chemical etching, stamping, impaction, pressing or molding. It can be achieved by removing, deforming, or adding biocompatible material, or by blasting with particles of bone or bone like material that adhere to or abrade bone (including those adherence models in which the particles have the desired macro- and microroughness without altering the bone surface itself).

In another alternative embodiment, the bone graft surface is adapted by fracturing, or splitting the bone graft through its body to generate two or more bone grafts each having a fracture surface. It is the fracture surface which is then implanted adjacent the host bone. The method of fracturing may include initiating a fracture using a means such as a chisel before pulling the bone graft pieces apart.

The invention also applies to natural bone, in vivo, whose healing and regeneration can be enhanced or promoted by applying a surface roughness similar to a surface roughness of a bone fracture surface. The preferred surface roughness is as defined previously for the bone graft 10 of the present invention.

A method for adapting bone for promoting bone regeneration comprises accessing at least a portion of a surface of bone to be adapted, the bone comprising bone material; and adapting the surface to produce a surface roughness similar to that of a fracture surface of bone. The method of adapting the bone surface includes selectively indenting bone material at the surface and/or selectively removing bone material from the surface whilst maintaining the structural integrity of the bone to achieve the desired the surface roughness or texture. This is preferably achieved by mechanically working or contacting selected portions across the surface with at least one impacting tip, or an array of impacting tips. The impacting tip or tips should have a diameter suitable for producing the desired roughness, preferably ranging from about 0.5 to about 25 μm. In a preferred embodiment, the surface is selectively worked across its surface by contacting the impact tip on different locations on the surface. It will be appreciated that by adapting the surface of the bone only, the removal of an excess of bone is avoided which minimizes trauma to the bone and maintains the structural integrity of substantially the whole bone. The surface being mechanically worked can be water cooled during or after mechanically working to further minimize damage and trauma to the bone.

The bone surface to be adapted may be the outer cortical surface of bone. For example, the cortical or periosteal surface around a fracture can be adapted as described, prior to applying a graft or other fixation means across the fracture. The roughened periosteal surface will have the effect of encouraging bone formation and regeneration around the bone fracture site and improved fixation of the graft or other fixation means across the fracture. The applied bone graft may also have a roughened surface contactable with the roughened host bone surface to further enhance bone formation at the interface of the bone graft and the host bone surface.

The bone surface to be adapted may include the inner cortical surface of bone. For example, in the case of a patient undergoing a hip replacement, a metal implant is usually placed into the femoral canal and may be fixed in place by bone cement. The use of bone cement may be avoided and the fixation of the implant to the inner cortical bone may be improved by applying the method of the present invention to at least a portion of the bone surface in the femoral canal (endosteal surface) to promote bone regeneration at that surface.

The bone surface to be adapted may be an intermediate bone surface such as a cut surface of cortical bone which may have been cut during a re-section or excision e.g. to remove a tumour or infection.

The invention also includes a use of a device to adapt a surface of a bone material, the device having an impact tip arranged to contact the surface of the bone material to adapt the surface to produce a surface roughness similar to the roughness or texture of a typical fracture surface of bone. The preferred surface roughness is as defined previously for the bone graft 10 of the present invention. The bone material may be a bone graft or natural bone in vivo.

In one embodiment, the impact tip is moveably attached to a powered hand tool for ease of handling and is arranged to be mechanically reciprocating with respect to the handle tool. The hand tool can be a Dremel™ engraver having a carbide impact tip of between 0.5 to 25 μm diameter. In use, the hand tool is moved across the bone material surface so that the impact tip can come in contact with different areas of the bone material surface to obtain the desired surface roughness. Without wishing to be bound by theory, it is thought that the action of the impact tip on the bone surface leads to impaction of some of the bone material and also removal of some of the bone material to create the surface roughness of bone. Three passes of the impact tip across the surface whilst rotating the surface 30° between passes has been found to be adequate to produce the required surface roughness.

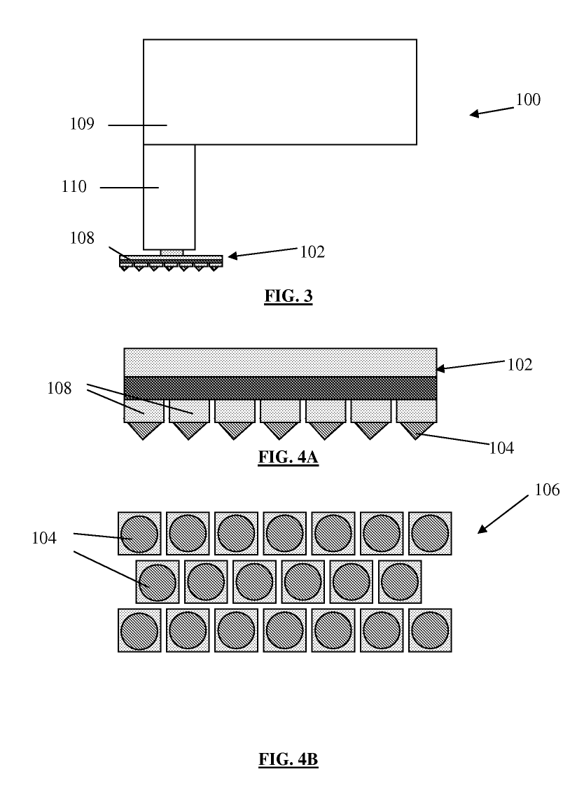

Referring now to FIGS. 3 and 4, there is shown a device 100 according to the present invention for adapting bone material. The device 100 comprises a first head 102 having a plurality of impact tips 104 arranged thereon as an array 106 to contact a surface of the bone material to be adapted to produce a bone material surface roughness similar to the roughness of a fracture surface of bone, as previously defined. The array of impact tips can be seen most clearly in FIGS. 4A and 4B.

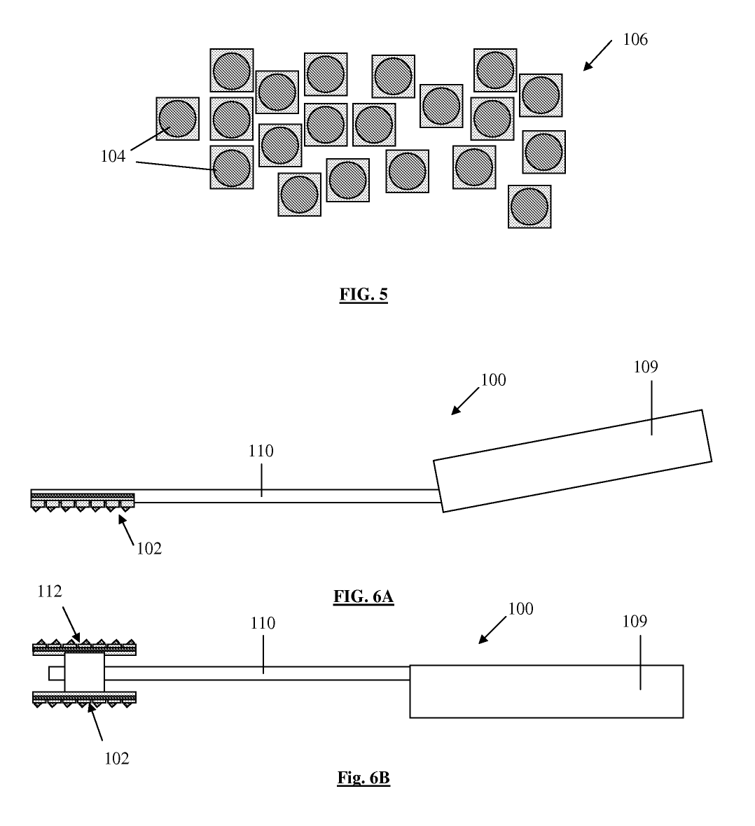

The impact tips 104 are sized and shaped to produce a bone material surface roughness similar to the roughness of a fracture surface of bone, as previously defined. Each impact tip 104 is made of any suitable material for roughening bone, such as carbide or diamond, and has a tip diameter of between about 0.5 and about 25 μm. The impact tips 104 may be arranged in an ordered manner (FIG. 4B). According to another embodiment of the head shown in FIG. 5, the impact tips 104 may be arranged in a non-ordered, random manner on the first head 102. The arrangement of the impact tips 104 in the array 106 may differ from as shown in the figures and described herein in ways which will be apparent to a person skilled in the art.

Referring back to FIG. 3, the device 100 further comprises an actuator 108 for providing a reciprocating movement to the impact tips. The actuator is piezoelectric, but may also be any other type of actuator, and is associated with each impact tip 104 to be able to control the movement of each impact tip 104 individually. Alternatively, there may be provided a common actuator (not shown) for driving the movement of the impact tips. Each impact tip 104 may be mounted to the first head 102 to allow relative movement between the impact tip 104 and the first head 102 to enable the impact tips to adapt to, and contact the contours to which it is being applied to. In another alternative embodiment, the actuator is arranged to drive the movement of the first head rather than the impact tips. In a yet further embodiment, the actuator may be remote to the device and movement of the head or the impact tips driven by another means.

The device 100 includes a handle 109 to which the first head 102 is connectable, to enable easy manipulation. The handle 109 may be a powered surgical tool. In this regard, the device 100 includes a first connector 110 for connecting the first head 102 to the handle 109. The first head 100 may be moveably, such as pivotably, connected to the handle 109.

In the embodiments shown in FIGS. 6A and 6B, the first connector 110 is an elongate arm having a first end to which the first head 102 is attachable, the elongate arm 110 being sized and shaped to access and contact surfaces of bone in vivo in a minimally invasive manner.

In the embodiment of the device 100 shown in FIG. 6B, the device 100 further comprises a second head 112 having a plurality of impact tips 104 arranged thereon as an array 106 to contact a surface of the bone material to be adapted to produce a bone material surface roughness similar to the roughness of a fracture surface of bone, the second head being attachable to the elongate arm 110. The device 100 of this embodiment is best suited to accessing and adapting internal bone surfaces such as the surface of the femoral canal. In this respect, the distance of the first and second heads from one another is not wider than the width of an average femoral canal. The impact tips of the first and second heads 102, 112 will reciprocate back and forth to impact different areas of a patient's femoral canal. Preferably, the distance between the first and second heads 102, 112 is adjustable to adapt the device for different sizes of femoral canal. In this respect, the first and second heads are moveable towards and away from each other whilst remaining attached to the elongate arm 110. In an alternative embodiment (not shown), the first and second heads are connected by a third connection means which is resiliently biased so that the first and second heads 102, 112, will move away from each other until they contact a surface such as the surfaces of the femoral canal. The first and second heads 102, 112 may also be pivotably moveable with respect to the elongate arm.

### EXAMPLES

The following examples are illustrative of the wide range of applicability of the present invention and are not intended to limit its scope. Modifications and variations can be made therein without departing from the spirit and scope of the invention. Although any method and material similar or equivalent to those described herein can be used in the practice for testing of the present invention, the preferred methods and materials are described.

The examples below demonstrate the Inventors' surprising discovery that some of the physical surface characteristics of bone fracture surfaces are substantially similar to one another regardless of the mode of fracture or the species from which the bone is derived. The examples also demonstrate that bone formation and bone healing is enhanced in the presence of bone disks (in vitro) and bone autografts and allografts (in vivo) having a surface texture substantially similar to that of bone fracture surfaces (textured). Specifically, the Inventors have found that mineralization and bone formation is enhanced in cultures of bone marrow derived mesenchymal cells (MSCs) grown on bone disks whose surfaces have been adapted to resemble the texture of a fracture surface of bone (referred to herein as “textured surfaces”), compared with those grown on polished disks which best represent endosteal or periosteal surfaces. The Inventors' in vivo models clearly demonstrate enhanced bone formation in the presence of textured endogenous and allograft cortical bone surfaces in the rabbit and xenograft cortical bone surfaces in the rat.

### Example 1

### Bone Fracture Mode does not Affect Surface Topography

A fracture model capable of generating commonly observed clinically observed long bone fracture types (butterfly, transverse, comminuted, oblique) was developed to determine the fracture surface morphology for each of these fracture types as well as for periosteal and endosteal bone. Twenty paired femurs from skeletally mature mongrel canines were obtained immediately following euthanasia. Canine femurs are known to approximate human bone physiology, healing and remodelling. The femurs were carefully stripped of soft tissue whilst preserving the periosteum and avoiding damage to the periosteal surface. The harvested femurs were immediately wrapped in saline soaked surgical towels, placed in self-locking bags and frozen at −20° C. until testing. Prior to testing, the paired femurs were thawed at room temperature for 2 hours.

The proximal and distal ends of each femur were fixed in polymethylmethacrylate (PMMA) to produce a constrained and regular surface for mounting in a fracture device. The fracture device comprised a servo hydraulic machine to provide and measure axial load and a pneumatic cylinder ram. The femur to be tested was mounted to the fracture device such that subsidence and axial rotation was avoided upon slight axial load, and there was some freedom of motion at both the proximal and distal femoral joints of the femur. Axial load, impact velocity, and cortical penetration were the controlled parameters for fracture generation, and each variable was limited to two experimental values: axial load was maintained at either 150N (Low) or 1000N (High), impact velocity was 5000 mm/sec (Low) or 9000 mm/sec (High) and cortical penetration spanned 1 (Low) or 2 (High) cortices. Axial load was controlled by the servo hydraulic tensile machine; impact velocity was controlled by manipulating air pressure; and adjustment of the pneumatic cylinder position controlled cortical penetration.

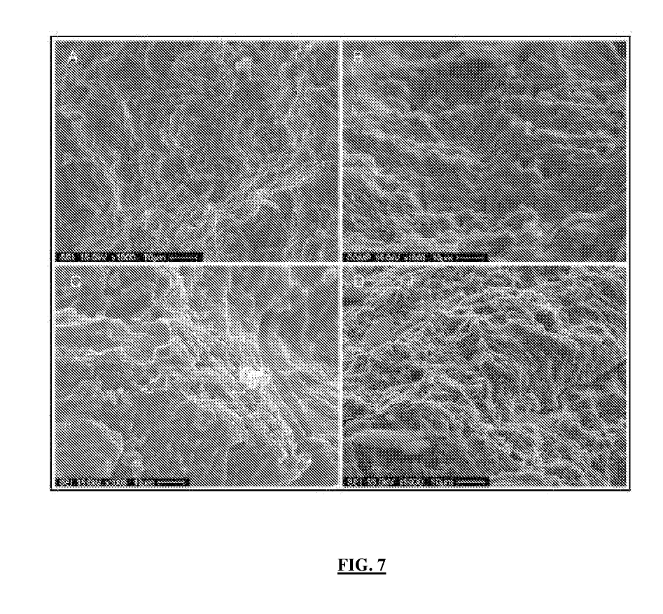

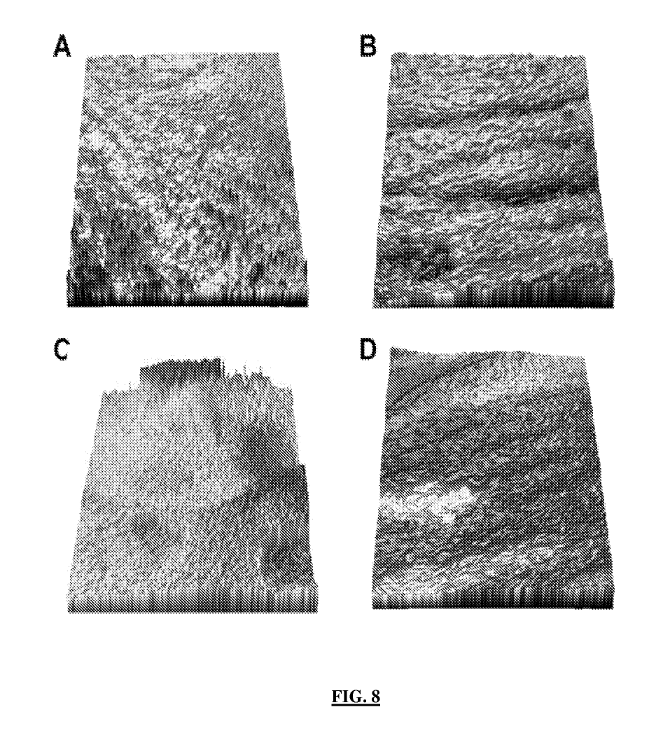

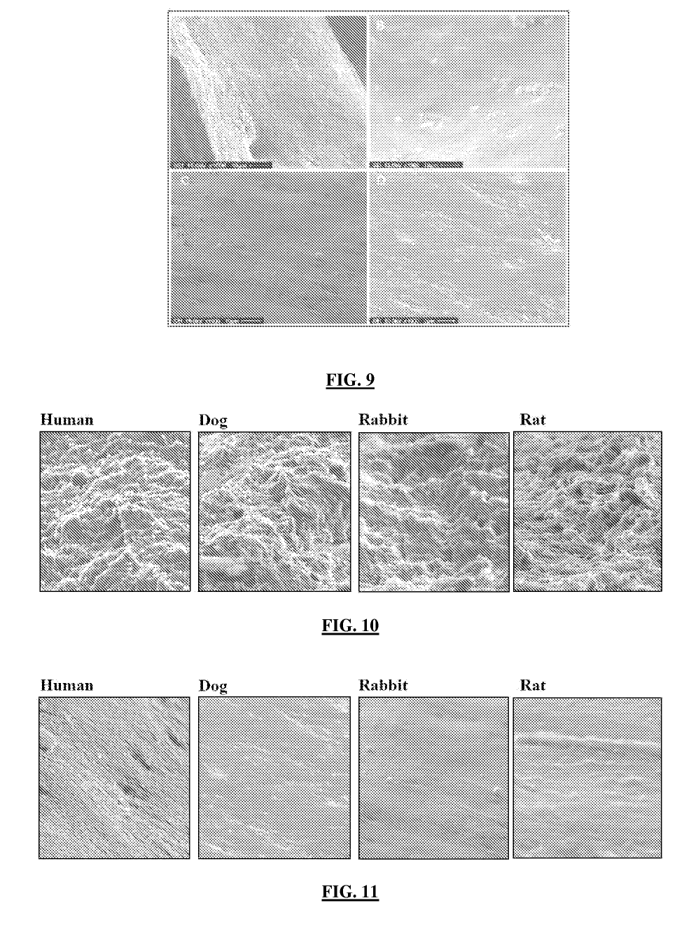

A qualitative impression of morphology of the fracture surfaces was obtained by scanning electron microscopy (FIGS. 7 and 9). Surface topography was quantified using a non-contact optical profiler (FIG. 8). The profiler was calibrated before use and the operational parameters were: VSI mode, 52× Mag, VSI filter, and tilt correction. The optical profiler is based on optical phase shifting and vertical scanning interference microscopy for surface analysis. This system permits a large vertical range measurement (0.1 nm to 2000 mm) with a resolution of Ra<1 Angstrom. Data is acquired digitally and processed by software that enables the determination of a number of parameters of interest: peak spacing, mean average centerline roughness, and peak height. Quantification included measurements of roughness including Rₐ (mean roughness) and R_{q} (root mean square roughness).

Scanning electron micrographs (FIG. 7) and three dimensional topographical scans (FIG. 8) demonstrated that the fracture surfaces, within the micron scale, comprised larger macrofeatures (such as peaks or waves across the surfaces) and smaller microfeatures which were superimposed on the surfaces of the macrofeatures. Overall, the fracture surfaces were not uniform in texture i.e. there were no repeated patterns such as grooves or striations. In other words, the arrangement of the macrofeatures was irregular across the surfaces. Most samples possessed a rough irregular texture with a clearly visible surface topography that was sometimes interspersed with areas of relatively smooth texture.

Table 1 illustrates the ranges and the mean values for Ra and Rq. Paired t-test analysis of the surface topographies of each fracture type showed that there was no significant difference in Ra and Rq between the different fracture types. Sample values were pooled and the overall roughness of all sample surfaces was calculated and determined to be Rₐ=2.85±1.32 μm and R_{q}=3.73±1.66 μm. Only two measurements had a roughness (Rₐ) less than 1.0 μm. There was no measured roughness (Rₐ) less than 0.5 μm for a bone fracture surface.

TABLE 1
Mean Ra and Rq values for each fracture surface
group (n = 36 per group).

| Bone fracture type | Mean Ra (μm) | Rq (μm) |
|---|---|---|
| Transverse | 2.66 ± 1.40 | 3.49 ± 1.74 |
| Butterfly | 2.90 ± 1.40 | 3.80 ± 1.87 |
| Oblique | 3.10 ± 1.33 | 4.11 ± 1.63 |
| Comminuted | 2.74 ± 1.02 | 3.52 ± 1.40 |
| Overall | 2.85 ± 1.32 | 3.73 ± 1.66 |

Overall values are an average of all types of fracture surfaces.

The peak-to-peak spacing, peak-to-valley height and peak diameter measurements of the bone fracture surfaces are illustrated in Table 2. As there was no significant difference between the Ra and Rq vales for the different fracture modes, the bone fracture surfaces from transverse mode fractures (the most common fracture mode) was studied only to measure the peak parameters of peak-to-peak spacing, peak-to-valley height and peak diameter values (Table 2). These were measured manually from SEM photographs at ×1000, 2000, 4000 magnifications. The increasingly higher SEM magnifications revealed different “layers” of textures superimposed on one another.

TABLE 2
Mean peak-to-peak spacing, peak-to-valley height and peak
diameter values for transverse fracture surface group

| Mag for transverse | Peak to peak | Peak to valley | Peak diameter |
|---|---|---|---|
| fracture | spacing (μm) | height (μm) | (μm) |
| ×1000 | 14.31 ± 5.26 | 10.41 ± 5.29 | 9.15 ± 2.32 |
| ×2000 | 4.37 ± 1.67 | 3.28 ± 0.83 | 2.28 ± 0.80 |
| ×4000 | 0.57 ± 0.04 | 0.79 ± 0.09 | 0.30 ± 0.03 |

In contrast, the endosteal and periosteal cortices (non-fractured surfaces) possessed a uniformly smooth surface texture in comparison to that of the fracture surfaces (FIG. 9). The endosteal cortex possessed an overall surface roughness of Rₐ=0.54±0.17 μm and R_{q} 0.78±0.28 μm, the periosteal cortex possessed an overall surface roughness of Rₐ 0.62±0.094 μm and R_{q} of 1.07±0.14 μm. The peak-to-peak spacing, peak-to-valley height and peak diameter values of these surfaces were measured manually from SEM photographs at ×250 and ×500 and averaged as there was no significant difference between the two magnifications (Table 3).

TABLE 3
Mean peak-to-peak spacing, peak-to-valley height and
peak diameter values for non-fractured bone surfaces

| Peak to peak | Peak to valley | Peak diameter |
|---|---|---|
| spacing (μm) | height (μm) | (μm) |
| 179 ± 71 | 1.08 ± 0.34 | 139 ± 60 |

### Example 2

### Animal Species does not Affect Bone Fracture Surface Topography

Analysis of the bone fracture surfaces from a number of species (mouse, rat, rabbit and canine) determined that the texture of the fracture surfaces did not vary significantly between species. Transverse fractures were created by placing the bones in a guillotine. The fracture surfaces had a distinct irregular texture (FIG. 10) and were as defined previously in Example 1. The fracture surfaces had an average roughness (Rₐ) of approximately 2.7-3.3 μm. In contrast, analysis of the endosteal and periosteal bone surfaces revealed a much “smoother” surface with an average roughness Rₐ of 0.4-0.6 μm (FIG. 11).

### Example 3

### Textured and Fractured Bone Surfaces Promote Osteoblast Mineralization In Vitro

An in vitro approach was used to assess the response of Canine marrow stromal cells (K9MC) cells to bone disks with surfaces which had been textured so that they were substantially similar to bone fracture surfaces, bone fracture surfaces and polished bone surfaces. Cultures were assessed for proliferation and evidence of mineralization.

Bone disks Bone disks (FIG. 12) were fabricated from bovine tibia. Bone plugs were cut perpendicular to the tibial shaft using a 21 mm (internal diameter) trephine bit The harvested bone plug was split with an osteotome in the direction of the long axis of the tibia to produce 90 halves each with a fractured surface (Fx bone). Sixty disks were further prepared by petrographic polishing to yield a smooth surface (Pol bone). Thirty of the polished disks were textured in a manner as defined in the present invention. Specifically, a Dremmel™ engraver having a 20 μm reciprocating tip was used to produce a textured surface. The bone disk was rotated by about 30° in between three passes of the engraver across each bone disk to ensure a random texture similar to that of a bone fracture surface. The bone disks were washed with distilled water and placed in a de-fatting solution (1:1 mixture of ether and acetone) for a period of 48 hours. Each disk was then removed from the de-fatting solution and soaked in distilled water for 48 hours before being dried in a fume hood for 24 hours. All culture disks were sterilized by gamma irradiation.

Surface Topography Scanning electron micrographs of the fractured and textured surfaces were obtained to provide a qualitative impression of surface morphology. Surface topography was quantified using a Wyko NT 2000 (Veeco, Rochester, N.Y.) noncontact optical profiler. The profiler was calibrated before use and the operational parameters were: VSI mode, 52 X Mag, VSI filter, and tilt correction. Three random regions from three disks of each group were analyzed yielding nine measurements per surface. Quantification included measurements of roughness including Rₐ (mean roughness).

Canine marrow stromal cells (K9MC) Using standard aseptic techniques, bone marrow was harvested from the iliac crest of a skeletally mature mongrel dog. Briefly, a single cell suspension was created by gently and repeatedly passing the suspension through a 21 gauge needle. Cells were then filtered through a 40 μm nylon filter and suspended in 20 ml PBS. Cells were counted and plated at a density of 65 million in P-100 dishes and cultured in αMEM supplemented with 10% FBS and 60 μg/ml Kanamyacin. Media was changed daily.

Cell Culture Cells were cultured on the bone disks or tissue culture plastic (TCP), for up to 43 days in αMEM supplemented with 10% FBS, 100 U/ml penicillin, 100 μg/ml streptomycin, 50 μg/ml ascorbate and 5 mM β-glycerophosphate in a 5% CO₂ air-balanced incubator at 37° C. Cells were seeded at a plating density of 30,000 cells per cm² or 125,000 cells per well. For each experiment, an additional set of TCP wells was maintained in the above media without the addition of β-glycerophosphate to provide a non-mineralizing control.

Cell Proliferation Cells were harvested after 1, 3, 6 and 12 days in culture to determine the increase in cell number. Cell proliferation was determined by quantification of total DNA according to the method of Labraca et al. Briefly, culture media was aspirated from the wells and disks were washed three times in PBS NaCl following which, cells were harvested in a solution of PBS 2M NaCl. In cases where a cell layer existed it was removed in its entirety, otherwise cells were removed by a combination of cell scraping and vigorous pipetting. Cells were kept on ice and lysed by sonification. DNA content was determined by Hoescht dye. The sample solutions were diluted as follows: 1 day—no dilution, 3 days—5 times dilution, 6 days—10 times dilution, 12 days—20 times dilution. Each sample received 35 μl of Hoechst dye and if the sample was diluted the balance to 965 μl was made up with PBS 2M NaCl.

Mineralization Assay The uptake of ⁴⁵Ca was used to determine the rate of mineralization. At day 43, samples were incubated for a period of 5 hours in α-MEM+10% FCS containing 0.5 μCi/ml ⁴⁵Ca followed by a 15 min incubation in α-MEM+10% FCS. The radioactive media was aspirated and the disks washed 3 times with 0.9% PBS NaCl and once with 1% H₂PO₄. A previous set of experiments had determined that ⁴⁵Ca bound non-specifically to bone and that a wash in 1% H₂PO₄ removed>98% of all non-specifically bound ⁴⁵Ca. The ⁴⁵Ca incorporated into the cells was released by dissolution in a 12.5% solution of tricarboxylic acid for a period of 12 hours at 4° C. The Ca⁴⁵ content from each sample was determined by scintillation counting for a 2 minute period. Four and a half ml of Ecolite™ was added to 0.8 ml of the sample.

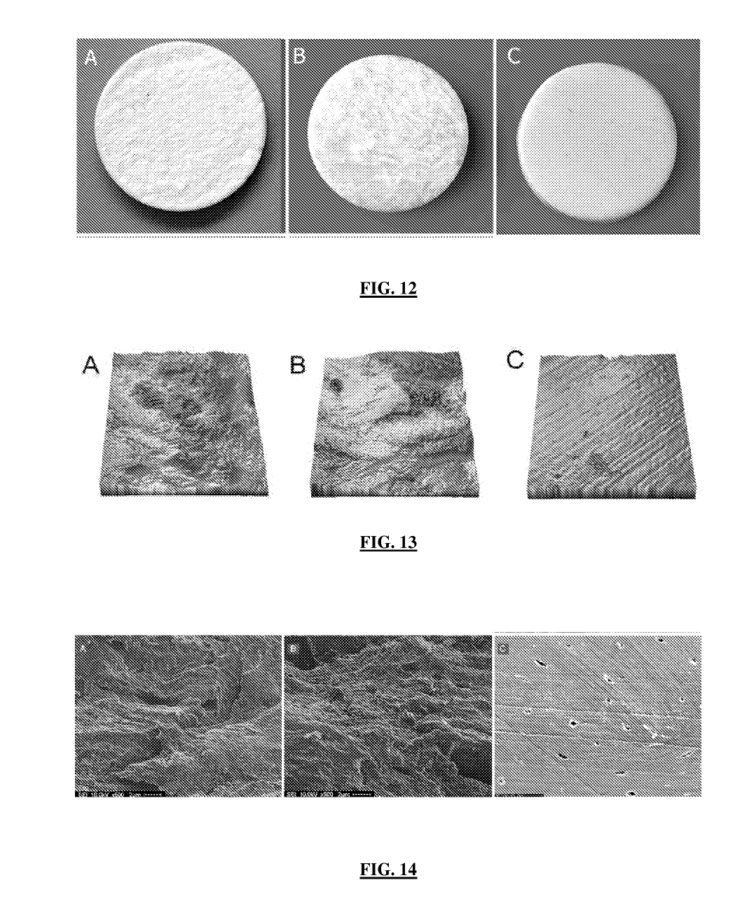

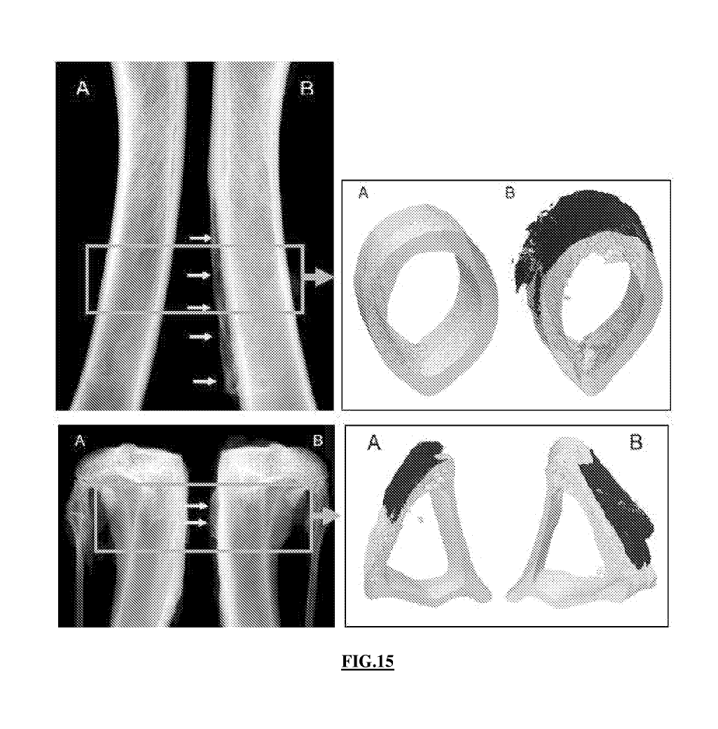

Results—Surface Topography Scanning electron micrographs of the culture disks (FIG. 14) showed an irregular texture on the Fx bone and textured bone surfaces which was comparable to that seen on the bone fracture surfaces reported in Examples 1 and 2. This irregular texture was not present on the Pol bone and TCP surfaces. The Pol bone and TCP surfaces were similar in appearance. However, depressions (intersection of vascular canals) were present in the Pol bone disks (FIG. 14C). Three dimensional topographical scans (FIG. 13) demonstrated that the Fx Bone and Tx bone surfaces presented an irregular texture as identified previously in Examples 1 and 2, whereas the Pol bone and TCP presented a relatively smooth surface texture. A significant difference in surface roughness (Ra) existed between the smooth and textured bone (p>0.05) and the smooth and fractured bone surfaces. The Rₐ of the Fx bone was not significantly different from that of the textured bone disks (Table 4).

TABLE 4
⁴⁵Ca uptake on polished and textured bone and titanium surfaces

| Surface Evaluated | Ra (μm) | cpm ⁴⁵Ca (×1000) |
|---|---|---|
| Fractured Bone (Fx bone) | 2.37 ± 1.14 | 442 ± 61 |
| Textured Bone (Tx bone) | 2.8 ± 1.1 | 420 ± 75 |
| Polished bone (Pol bone) | 0.1 ± 0.0 * | 6.6 ± 1 |
| Tissue Culture Plastic (TCP) | 0.01 ± 0.0 | 6 ± 1 |
| Tissue Culture Plastic (TCP -BGP) ** | 0.01 ± 0.0 | 6 ± 1 |

* Intersection of vascular canals at polished bone surface increased Rₐ
** Negative control without addition of BGP. Indicates background level of ⁴⁵Ca in culture where no mineralization occurs.

The peak parameter values of the macroroughness were measured manually from SEM photographs as before (Table 5).

TABLE 5
Average peak-to-peak spacing, peakto-valley height
and peak diameter for the textured bone disks

| Mag for transverse | Peak to peak | Peak to valley | Peak diameter |
|---|---|---|---|
| fracture | spacing (μm) | height (μm) | (μm) |
| ×1000 | 27.75 ± 8.07 | 13.72 ± 4.65 | 11.64 ± 2.15 |
| ×2000 | 6.85 ± 2.13 | 4.19 ± 0.83 | 4.48 ± 1.12 |

Results—Cell Proliferation Cells proliferated on all surfaces. Cell populations were greater on the smooth surfaces (Pol Bone and TCP) than the textured or fractured bone surfaces at all time points. This difference was significant (P<0.05) at days 3, 6 and 12. At approximately day 21 the visible cell layer detached from the smooth surfaces and formed a spherical mass. A continuous cell layer was also visible on the rough surfaces at this time.

Results—Mineralization Uptake of ⁴⁵Ca occurred on the fractured and textured surfaces only and was not significantly different. Significantly lower amounts of ⁴⁵Ca were uptake (p<0.05) occurred on the smooth surfaces, which was consistent with levels associated with background noise from non-mineralizing controls.

### Example 4

### Osseous Response to Cortical Surface Microtexturing

The periosteum was surgically retracted from a 10×30 mm region of bone on the distal femur and proximal anterior tibia of young adult rabbits to expose the underlying bone. An irregular texture (Rₐ of 3-4 μm) resembling that of a fracture surface of bone was applied to the smooth cortical bone using an embodiment of the method according the present invention. Specifically, a reciprocating impact tip of a Dremmel™ engraver was used to selectively impact and remove material from the cortical bone surface to create the required surface texture. A reproducible texture, as measured with white light interferometry, was obtained in this manner on in vivo cortical surfaces. The contra-lateral femur and tibia underwent a similar procedure but without cortical texturing. The experiment was terminated 6 weeks later and new bone formation quantified by micro CT after radiographic analysis. The application of a texture to cortical bone resulted in a distinct region of new bone formation, approximately 1.5 mm in height that corresponded closely with the textured region. Compared to the contralateral side that underwent a sham surgery, a 4-8 fold increase in bone formation was observed on the textured side of 6 samples (FIG. 15).

### Example 5

### Texturing of Xenograft Bone Enhances Bone Formation

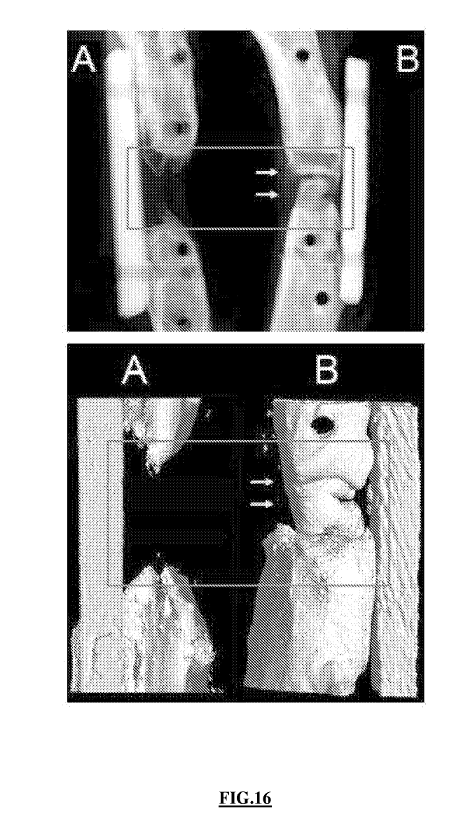

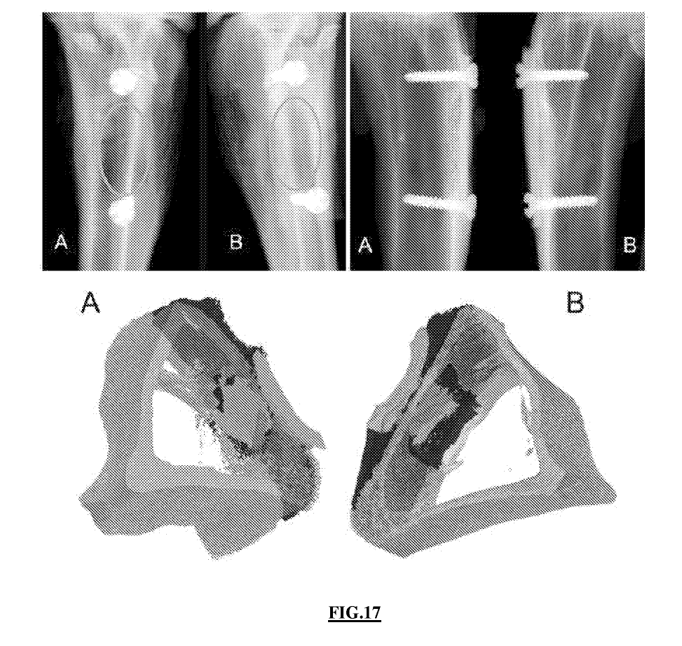

The impact on new bone formation of texturing relatively smooth bone to simulate the texture of a bone fracture surface was also assessed in a pilot study in rats that utilized a 5-mm segmental femoral defect model and bovine xenografts with either polished or textured surfaces. Data from 4 femoral pairs indicated a four-fold (20% Sm vs 80% Tx) increase in new bone formation, evaluated by quantitative mCT, on and within the defect site in the presence of a textured graft (FIG. 16). Bovine bone was used from the bone graft in this model because the small size of the rat bone was poorly suited for texturing with the device of one of the embodiments of the present invention. A surface of the bovine bone xenograft was textured in a manner and using a device according to one embodiment of the present invention. These results demonstrate that the presence of textured, devitalized bone in the defect area can be a stimulus for new bone formation. It is hypothesized that due to the absence of osteoprogenitor cells in the de-vitalized allograft, they must have arisen from the sub-periosteal region and/or from the marrow cavity.

### Example 6

### Textured Allografts Stimulate New Bone Formation

Another model was designed to examine the influence on bone formation of applying a texture similar to the texture of bone fracture surfaces to cortical allografts. A cortical window of 5 mm×10 mm with direct access to the tibial canal was created in the proximal anterior tibia of rabbits after incising the periosteum (FIG. 17). A cortical allograft, large enough to occlude the window was positioned and fixed securely in place with screws before closing the periosteum and wound. The contra-lateral tibia underwent a similar procedure but without allograft texturing. In keeping with the results from the endogenous bone texturing described above, bone regeneration in the cortical window was enhanced 2.5 fold in the presence of the textured allograft. Please note that this response was observed in relatively short (6 week) duration. Allografts were textured on both sides and soft tissue attachment was found on the textured side opposite to the side opposing bone.

While several embodiments of the invention have been described herein, it will be understood that the present invention is capable of further modifications, and this application is intended to cover any variations, uses, or adaptations of the invention, following in general the principles of the invention and including such departures from the present disclosure as to come within knowledge or customary practice in the art to which the invention pertains, and as may be applied to the essential features hereinbefore set forth and falling within the scope of the invention as defined in the appended claims.

It should be appreciated that the invention is not limited to the particular embodiments described and illustrated but includes all modifications and variations falling within the scope of the invention as defined in the appended claims.

1. A method for adapting a bone graft implantable into a host bone for promoting host bone regeneration, the method comprising:
providing a bone graft comprising bone material and having a surface contactable with a host bone; and
adapting at least a portion of the surface to produce a surface roughness similar to the roughness of a fracture surface of bone.

2. A method according to claim 1, wherein the bone graft surface is adapted to include macrostructures and microstructures, the macrostructures being peaks and having a peak-to-peak spacing which is substantially less than that of an unfractured bone surface.

3. A method according to claim 2, wherein the peak-to-peak spacing is less than about 180 μm.

4. A method according to claim 1, wherein the bone graft surface is adapted by selectively indenting bone material at the surface or selectively removing bone material from the surface.

5. A method according to claim 1, wherein the bone graft surface is adapted by mechanically contacting the surface with at least one impacting tip to adapt it.

6. A method according to claim 1, wherein the bone surface is adapted by mechanically contacting the surface with a plurality of impacting tips arranged as an array, and changing the positioning of the bone graft surface with respect to the array of impacting tips in between contacting to create an irregular surface roughness.

7. A bone graft having a surface contactable with a host bone for promoting regeneration of the host bone, wherein at least a portion of the surface has a surface roughness similar to that of a fracture surface of bone.

8. A bone graft according to claim 7, wherein the bone graft surface includes macrostructures and microstructures, the macrostructures being peaks and having a peak-to-peak spacing which is substantially less than that of an unfractured bone surface.

9. A bone graft according to claim 8, wherein the peak-to-peak spacing is less than about 180 μm.

10. A bone graft according to claim 8, wherein the peak-to-peak spacing is between about 0.1 and about 180 μm.

11. A bone graft according to claim 7, wherein the surface roughness, as defined by Ra, is between about 0.1 and 400 μm.

12. A bone graft according to claim 7, wherein the bone graft surface includes macrostructures and microstructures, the macrostructures being peaks which are randomly distributed across the bone graft surface.

13. A method for adapting bone for promoting bone regeneration, the method comprising:
accessing at least a portion of a surface of bone to be adapted, the bone comprising bone material; and
adapting the surface to produce a surface roughness similar to the roughness of a fracture surface of bone.

14. A method according to claim 13, wherein the bone surface is adapted by selectively indenting bone material at the surface or selectively removing bone material from the surface.

15. A method according to claim 13, wherein the bone surface is adapted by mechanically contacting the surface with at least one impacting tip to adapt it.

16. A method according to claim 13, wherein the bone surface is adapted to include macrostructures and microstructures, the macrostructures being peaks and having a peak-to-peak spacing which is substantially less than that of an unfractured bone surface.

17. A method according to claim 16, wherein the peak-to-peak spacing is less than about 180 μm.

18. A method according to claim 13, wherein the surface is adapted to have a roughness, as defined by Ra, of between about 0.1 and about 400 μm.

19. A device for adapting bone material, the device comprising:
a first head having a plurality of impact tips arranged thereon as an array to contact a surface of the bone material to be adapted to produce a bone material surface roughness similar to the roughness of a fracture surface of bone.

20. A device according to claim 19, further comprising an actuator to provide a reciprocating movement to the plurality of impact tips.

21. A device according to claim 19, further comprising an elongate arm having a first end to which the first head is attachable, the elongate arm being sized and shaped to access an inner surface of the bone.

22. A device according to claim 21, further comprising a second head having a plurality of impact tips arranged thereon as an array to contact a surface of the bone material to be adapted to produce a bone material surface roughness similar to the roughness of a fracture surface of bone, the second head being attachable to the elongate arm.

23. A device according to claim 22, wherein the first and second heads are arranged to be moveable towards and away from each other whilst remaining attachable to the elongate arm.

24. A device according to claim 19, wherein the impact tips have a diameter of from about 0.5 μm to about 25.0 μm.

25. Use of a device to adapt a surface of a bone material, the device having an impact tip arranged to contact the surface of the bone material to adapt the surface to produce a surface roughness similar to the roughness of a fracture surface of bone.

26. Use according to claim 25, wherein the impact tip has a diameter of from about 0.5 μm to about 25.0 μm.

27. Use according to claim 26, wherein the impact tip has a diameter of about 20.0 μm.

28. Use according to claim 25, wherein the device further comprises an actuator to provide a reciprocating movement to the impact tip.

29. Use according to claim 25, wherein the device further comprises an elongate arm having a first end to which the head is attachable, the elongate arm being sized and shaped to access an inner surface of the bone.

30. Use according to claim 25, wherein the device further comprises a plurality of impact tips.
